(19) United States
(12) Patent Application Publication
(10) Pub. No.: US 2024/0225529 A1
(43) Pub. Date: Jul. 11, 2024

(54) SEM Trend Analysis

(71) Applicant: Bruin Biometrics, LLC, Los Angeles, CA (US)

(72) Inventor: Martin F. BURNS, Los Angeles, CA (US)

(73) Assignee: Bruin Biometrics, LLC, Los Angeles, CA (US)

(21) Appl. No.: 18/612,918

(22) Filed: Mar. 21, 2024

Related U.S. Application Data
(63) Continuation of application No. 16/213,664, filed on Dec. 7, 2018.
(60) Provisional application No. 62/596,089, filed on Dec. 7, 2017.
(60) Provisional application No. 62/623,857, filed on Jan. 30, 2018.

(51) Int. Cl. A61B 5/00

(52) U.S. Cl. CPC A61B 5/443; A61B 5/445; A61B 5/447; A61B 5/7275; A61B 5/7282

(57) ABSTRACT

The present disclosure provides for, and includes, a method of detecting tissue damage before it is visible on a patient's skin, comprising: measuring a plurality of sub-epidermal moisture (SEM) values at a single location at incremental times, calculating a slope between the latest SEM value and the immediately prior SEM value, comparing this slope to a threshold value, and determining that there is tissue damage if the slope exceeds the threshold value.

### CROSS REFERENCE TO RELATED APPLICATIONS

This application is a continuation of U.S. patent application Ser. No. 16/213,664 filed Dec. 7, 2018, which claims the benefit of U.S. provisional patent application Ser. Nos. 62/596,089 filed on Dec. 7, 2017, and 62/623,857 filed on Jan. 30, 2018, each of which are incorporated by reference in their entireties herein.

### FIELD

The present disclosure provides methods and apparatus for detecting tissue damage through evaluation of trends in Sub-epidermal Moisture (SEM) values.

### BACKGROUND

The skin is the largest organ in the human body. It is readily exposed to different kinds of damages and injuries. When the skin and its surrounding tissues are unable to redistribute external pressure and mechanical forces, ulcers may be formed. Prolonged continuous exposure to even modest pressure, such as the pressure created by the body weight of a supine patient on their posterior skin surfaces, may lead to a pressure ulcer.

### SUMMARY

In an aspect, the present disclosure provides for, and includes, a method of detecting tissue damage before it is visible on a patient's skin, comprising: measuring a plurality of sub-epidermal moisture (SEM) values at a single location at incremental times, calculating a slope between the latest SEM value and the immediately prior SEM value, comparing this slope to a threshold value, and determining that there is tissue damage if the slope exceeds the threshold value.

In an aspect, the present disclosure provides for, and includes, a method of detecting tissue damage before it is visible on a patient's skin, comprising: measuring a plurality sub-epidermal moisture (SEM) values at a plurality of locations at incremental times, calculating a delta value for the plurality of SEM values for each time, calculating a slope between the latest delta value and the immediately prior delta value, comparing this slope to a threshold value, and determining that there is tissue damage if the slope exceeds the threshold value.

In an aspect, the present disclosure provides for, and includes, a method of detecting tissue damage before it is visible on a patient's skin, comprising: measuring a plurality of sub-epidermal moisture (SEM) values at a single location at each of a plurality of incremental times, calculating a SEM delta value for each incremental time, fitting a curve to a predetermined number of the most-recent SEM delta values, calculating a curvature of the fitted curve, comparing this curvature to a threshold value, and determining that there is tissue damage if the curvature exceeds the threshold value.

### BRIEF DESCRIPTION OF THE DRAWINGS

Aspects of the disclosure are herein described, by way of example only, with reference to the accompanying drawings. With specific reference now to the drawings in detail, it is stressed that the particulars shown are by way of example and are for purposes of illustrative discussion of aspects of the disclosure. In this regard, the description and the drawings, considered alone and together, make apparent to those skilled in the art how aspects of the disclosure may be practiced.

FIGS. 1A, 1B, and 1C illustrate a progression of tissue damage toward a pressure ulcer.

FIG. 2 depicts example locations for SEM measurements on the sacrum, in accordance with the present disclosure.

FIG. 3 depicts SEM values over time for patients that do and do not develop pressure ulcers, in accordance with the present disclosure.

FIG. 4 depicts SEM delta values over time for patients that develop pressure ulcers, in accordance with the present disclosure.

FIG. 5 depicts illustrative SEM values and delta values over time for patients that develop pressure ulcers, in accordance with the present disclosure.

FIG. 6 depicts example SEM delta values over time for patients that develop pressure ulcers in the heels, in accordance with the present disclosure.

### DETAILED DESCRIPTION

This description is not intended to be a detailed catalog of all the different ways in which the disclosure may be implemented, or all the features that may be added to the instant disclosure. For example, features illustrated with respect to one embodiment may be incorporated into other embodiment, and features illustrated with respect to a particular embodiment may be deleted from that embodiment. Thus, the disclosure contemplates that in some embodiments of the disclosure, any feature or combination of features set forth herein can be excluded or omitted. In addition, numerous variations and additions to the various embodiments suggested herein will be apparent to those skilled in the art in light of the instant disclosure, which do not depart from the instant disclosure. In other instances, well-known structures, interfaces, and processes have not been shown in detail in order not to unnecessarily obscure the invention. It is intended that no part of this specification be construed to effect a disavowal of any part of the full scope of the invention. Hence, the following descriptions are intended to illustrate some particular embodiments of the disclosure, and not to exhaustively specify all permutations, combinations and variations thereof.

Unless otherwise defined, all technical and scientific terms used herein have the same meaning as commonly understood by one of ordinary skill in the art to which this disclosure belongs. The terminology used in the description of the disclosure herein is for the purpose of describing particular aspects or embodiments only and is not intended to be limiting of the disclosure.

All publications, patent applications, patents and other references cited herein are incorporated by reference in their entireties for the teachings relevant to the sentence and/or paragraph in which the reference is presented. References to techniques employed herein are intended to refer to the techniques as commonly understood in the art, including variations on those techniques or substitutions of equivalent techniques that would be apparent to one of skill in the art.

U.S. patent application Ser. No. 14/827,375 discloses an apparatus that uses radio frequency (RF) energy to measure the sub-epidermal capacitance using a bipolar sensor, where the sub-epidermal capacitance corresponds to the moisture content of the target region of skin of a patient. The '375 application also discloses an array of these bipolar sensors of various sizes.

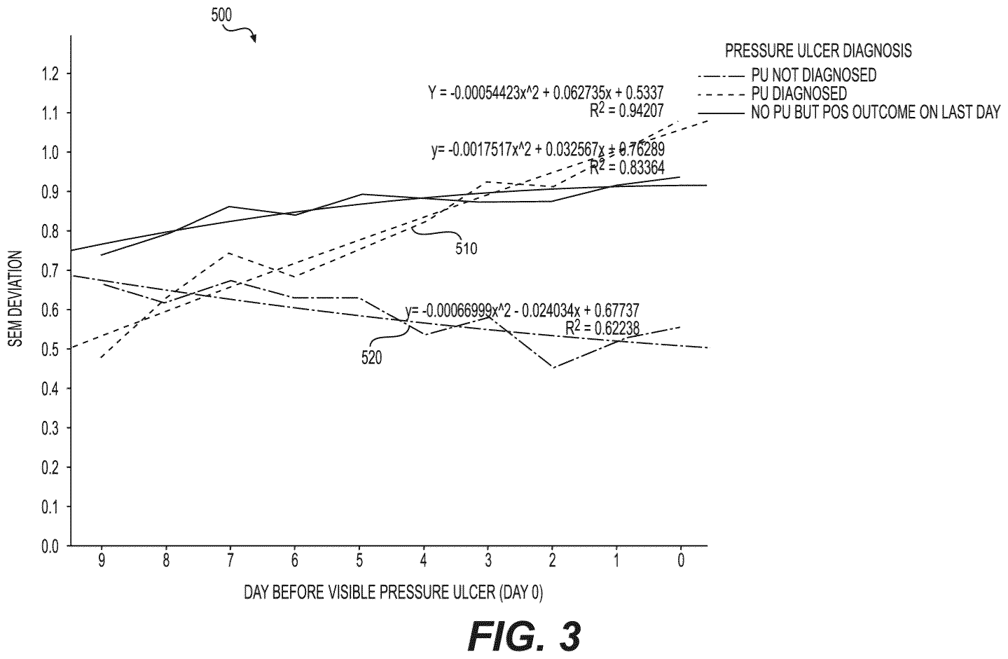

U.S. patent application Ser. No. 15/134,110 discloses an apparatus for measuring sub-epidermal moisture (SEM) similar to the device shown in FIG. 3, where the device emits and receives an RF signal at a frequency of 32 kHz through a single coaxial sensor and generates a bioimpedance signal, then converts this signal to a SEM value.

Both U.S. patents application Ser. Nos. 14/827,375 and 15/134,110 are incorporated herein by reference in their entireties. However, the SEM values of this application may be measured by any similar or equivalent devices or techniques that would be apparent to one of skill in the art. For example, a device measuring the SEM values of this application may be a wired device, a wireless device, or a system comprising various components in communication with each other.

Unless the context indicates otherwise, it is specifically intended that the various features of the disclosure described herein can be used in any combination. Moreover, the present disclosure also contemplates that in some embodiments of the disclosure, any feature or combination of features set forth herein can be excluded or omitted.

The methods disclosed herein include and comprise one or more steps or actions for achieving the described method. The method steps and/or actions may be interchanged with one another without departing from the scope of the present disclosure. In other words, unless a specific order of steps or actions is required for proper operation of the embodiment, the order and/or use of specific steps and/or actions may be modified without departing from the scope of the present disclosure.

As used in the description of the disclosure and the appended claims, the singular forms “a,” “an” and “the” are intended to include the plural forms as well, unless the context clearly indicates otherwise.

As used herein, “and/or” refers to and encompasses any and all possible combinations of one or more of the associated listed items, as well as the lack of combinations when interpreted in the alternative (“or”).

The terms “about” and “approximately” as used herein when referring to a measurable value such as a length, a frequency, or a SEM value and the like, is meant to encompass variations of ±20%, ±10%, ±5%, ±1%, ±0.5%, or even ±0.1% of the specified amount.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

As used herein, the term “sub-epidermal moisture” or “SEM” refers to the increase in tissue fluid and local edema caused by vascular leakiness and other changes that modify the underlying structure of the damaged tissue in the presence of continued pressure on tissue, apoptosis, necrosis, and the inflammatory process.

As used herein, a “patient” may be a human or animal subject.

As used herein, “delta” refers to a calculated difference between two SEM values.

There may be many causes of saddle sores, plenty of confounding elements, and a lot of closely inter-related issues behind the development of a saddle sore. They strike young and old, male or female, amateur or professional without provocation or distinction. Lance Armstrong received a “therapeutic use exemption” for cortisone in 1999, which had shown up in drug screening. Other professional riders have been forced to not compete due to ulcer-related injuries, although it is generally kept very quiet.

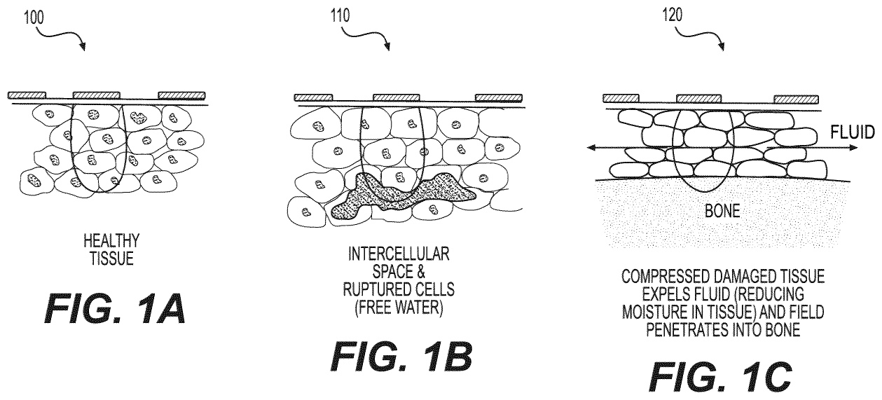

FIGS. 1A-1C illustrate a progression of tissue damage toward a pressure ulcer.

FIG. 1A depicts a cross-section of healthy tissue 100, including the stratum corneum and healthy cells in the epidermis/dermis. The center electrode and the toroidal electrode of a SEM scanner are shown in cross-section in contact with the stratum corneum. An illustrative indication of the sensitive region of the SEM Scanner is shown as the oval region. The region has a depth of sensitivity. In some instances, the depth of sensitivity is in the range of 0.14 to 0.16 inches. In some instances, the depth of sensitivity is less than 0.16 inches.

FIG. 1B is an illustrative cross-section of slightly damaged tissue 110. Cellular damage, for example resulting from long-term application of low-level pressure has affected the tissue. Without being limited by theory, some of the cells have ruptured, releasing the fluid contents into an intercellular space. Alternatively, and without being limited by theory, an inflammatory reaction has caused fluid to migrate into the intercellular space. This damage is not visible on the skin surface.

FIG. 1C is an illustrative cross-section 120 of a more advanced level of damage. Without being limited by theory, the tissue is now mostly ruptured cells, which can provide little mechanical structure to carry the continued applied pressure. The tissue thickness is reduced, with the bone now closer to the skin surface. The ruptured cells and intercellular space are compressed, expelling the fluid out of the local tissue as indicated by arrows.

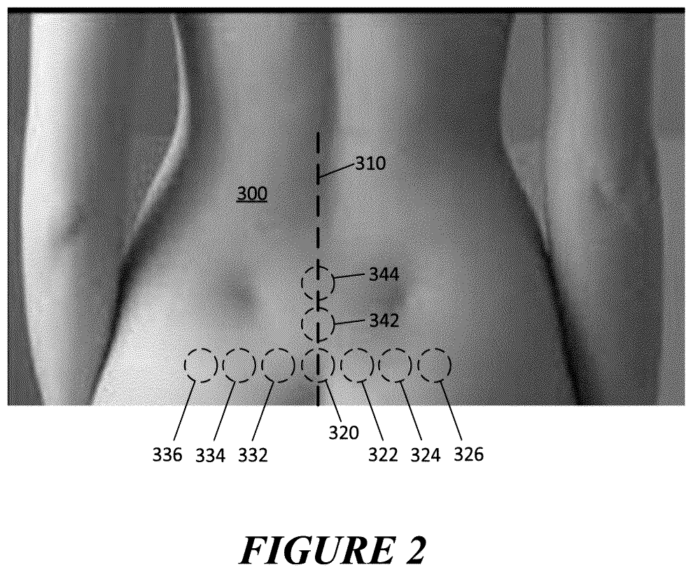

FIG. 2 depicts example locations for SEM measurements on the sacrum, in accordance with the present disclosure. In an aspect, SEM measurements can be taken around the center positioned at top of gluteal crease 320. In one aspect, centerline 310 can be established from the top of gluteal crease 320. In an aspect, SEM measurements can be taken at locations along centerline 310, for example, at locations 342 and 344. In one aspect, pairs of symmetric SEM measurements can be taken at locations approximately symmetric about centerline 310, for example, at locations 332 and 322, at locations 334 and 324, at locations 336 and 326.

In an aspect, a SEM value is a single SEM measurement. In an aspect, a SEM value is an average SEM measurement generated from SEM measurement values taken at approximately the same location on a patient's skin within a 24-hour period, such as within a 18-hour period, within a 12-hour period, within a 8-hour period, within a 6-hour period, within a 4-hour period, within a 3-hour period, within a 2-hour period, within an hour, within 45 minutes, within 30 minutes, within 15 minutes, within 10 minutes, within 5 minutes, within 1 minute, or within 30 seconds.

FIG. 3 depicts SEM values over time for patients that do and do not develop pressure ulcers, in accordance with the present disclosure. Curve 510 represents average SEM values for the days leading up to a diagnosis of a pressure ulcer on Day 0. The overlaid straight line is a linear approximation. Curve 520 represents average SEM values for the days leading up to a similar set of patients who did not develop a pressure ulcer. In both cases, there was no sign of damage or indication of a future pressure ulcer on the skin. The SEM values were indicative of subsurface damage that was invisible to visual and tactile examination. The overlaid straight line is a linear approximation.

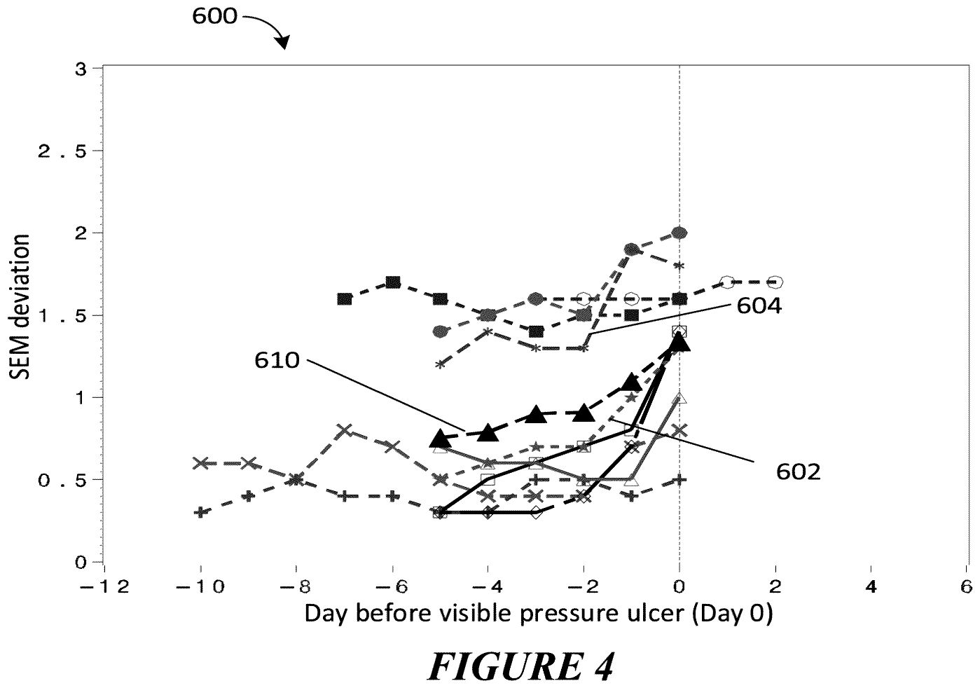

FIG. 4 depicts SEM delta values over time for patients that develop pressure ulcers, in accordance with the present disclosure. Curves 602 and 604 illustrate the acceleration of the rate of increase, i.e. the slope, of the curve as time gets closer to the point at which a visual examination leads to a clinical diagnosis. Curve 610 is an average of the other curves and shows the upward curve, i.e. acceleration of the rate of increase.

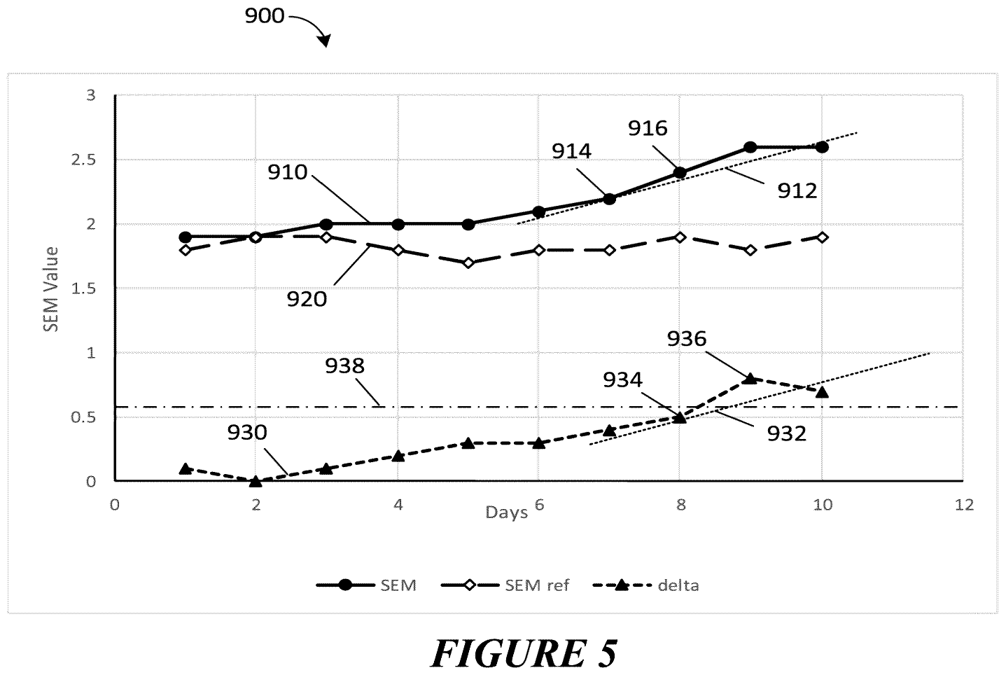

FIG. 5 is an example plot of measured and computed SEM values, in accordance with the present disclosure. Curve 910 is a set of SEM values for a skin area that is prone to development of a pressure ulcer. Curve 920 is a matching set of SEM values for a second skin area that is near the first area but not at risk for a pressure ulcer. Curve 920 serves as a reference. Curve 930 is a “delta” SEM value calculated by subtracting the reference value of curve 920 from the matching SEM value of curve 910.

Tissue damage may be detected in several ways. In one aspect, the slope of the SEM curve 910, for example the slope between points 914 and 916, is compared against a threshold slope, indicated by line 912. If the slope of the curve 910 exceeds the slope of line 912, this indicates a degree of damage. There may be multiple slopes used to evaluate multiple degrees of tissue damage. In one aspect, a slope is determined with respect to any two points on SEM curve 910, and is compared to the slope of line 912 to indicate a degree of damage. In an aspect, the slope of line 912 is determined by the health history of the subject. In one aspect, the curvature of a SEM curve is compared to a threshold curvature, where an over-curvature indicates a degree of damage.

In an aspect, the value of the delta curve 930 is compared to a threshold level 938. When curve 930 exceeds threshold 938, for example at point 936, this indicates a degrees of damage. There may be multiple thresholds used to evaluate multiple levels of tissue damage.

In an aspect, a threshold may be about 0.3, 0.35, 0.4, 0.45, 0.5, 0.55, 0.6, 0.65, 0.7, 0.75, 0.8, 0.85, 0.9, 0.95, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, or 7.5. In one aspect, a threshold may range from 0.1 to 8.0, such as from 0.1 to 1.0, from 1.1 to 2.0, from 2.1 to 3.0, from 3.1 to 4.0, from 4.1 to 5.0, from 5.1 to 6.0, from 6.1 to 7.0, from 7.1 to 8.0, from 0.1 to 7.5, from 0.5 to 8.0, from 1.0 to 7.0, from 1.5 to 6.5, from 2.0 to 6.0, from 3.0 to 5.5, from 3.5 to 5.0, or from 4.0 to 4.5. In an aspect, a threshold can be scaled by a factor or a multiple based on the values provided herein. It will be understood that a threshold is not limited by design, but rather, one of ordinary skill in the art would be capable of choosing a predetermined value based on a given unit of SEM. In one aspect, thresholds of the present disclosure are varied according to the specific portion of a patient's body on which measurements are being made, or one or more characteristics of the patient such as age, height, weight, family history, ethnic group, and other physical characteristics or medical conditions.

In one aspect, the slope of the delta curve 930, for example the slope between points 934 and 936, is compared against a threshold slope, indicated by line 932. If the slope of the curve 930 exceeds the slope of line 932, this indicates a degree of damage. There may be multiple slopes used to evaluate multiple degrees of tissue damage. In one aspect, a slope is determined with respect to any two points on delta curve 930, and is compared to the slope of line 932 to indicate a degree of damage. In an aspect, the slope of line 932 is determined by the health history of the subject. In one aspect, the curvature of a delta curve is compared to a threshold curvature, where an over-curvature indicates a degree of damage.

In an aspect, a SEM delta value above a predefined threshold value is an indication of sub-epidermal damage that may lead to a pressure ulcer. The time interval between the time when the SEM delta value first equals or exceeds this threshold and the development of visible symptoms of a pressure ulcer may be a first duration when the SEM delta value increases linearly. A first duration may be 5 or more days, such as 6 or more days, 7 or more days, 8 or more days, 9 or more days, or 10 or more days.

In another aspect, when the SEM delta curve shows an upward curvature or other deviation above a linear progression, the visible symptoms may be present within a shorter amount of time, for example 2-3 days, 1-4 days. 1-3 days, 1-2 days, or 2-4 days. In an aspect, the SEM scanner, which includes an SEM sensor and electronics to measure the capacitance of the SEM sensor and convert this measured capacitance to a SEM value and store a plurality of these SEM values then calculate and display a SEM delta value from the plurality of SEM values and transmit a portion of the measurements and delta values to a remote computer, is used to generate a SEM delta value for a particular location on the patient's skin, for example the heel. These SEM delta values are tracked and the trend of the SEM delta values, i.e. the slope and curvature of a curve connecting these SEM delta values, is analyzed. In an aspect, the amount by which an incremental SEM delta value is above a linear prediction based on prior SEM delta values is compared to a predetermined threshold. In an aspect, the amount by which an incremental SEM delta value is above the most recent prior SEM delta value is compared to a predetermined threshold. In an aspect, a curvature of the best-fit curve fitted to a predefined number of the most-recent SEM delta values is compared to a predetermined threshold. In an aspect, the number of sequential SEM delta values that exceeds a predetermined value threshold is compared to a number-of-readings threshold. In each of these aspects, the SEM scanner provides a notification when the comparison parameter exceeds the respective threshold.

In an aspect, the trend analysis may ignore a single SEM delta value that is below a threshold if both the prior and subsequent SEM delta values are above the threshold.

In an aspect, the trend curve of the SEM delta values is a point-to-point linear connection. In an aspect, the trend curve is a best-fit curve fitted to the SEM delta values. In an aspect, the fitted curve is required to intersection the most-recent SEM delta value.

From the foregoing, it will be appreciated that the present disclosure can be embodied in various ways, which include but are not limited to the following:

Embodiment 1. A method of detecting tissue damage before it is visible on a patient's skin, comprising: measuring a plurality of sub-epidermal moisture (SEM) values at a single location at incremental times, calculating a slope between the latest SEM value and the immediately prior SEM value, comparing this slope to a threshold value, and determining that there is tissue damage if the slope exceeds the threshold value.

Embodiment 2. A method of detecting tissue damage before it is visible on a patient's skin, comprising: measuring a plurality sub-epidermal moisture (SEM) values at a plurality of locations at incremental times, calculating a delta value for the plurality of SEM values for each time, calculating a slope between the latest delta value and the immediately prior delta value, comparing this slope to a threshold value, and determining that there is tissue damage if the slope exceeds the threshold value.

Embodiment 3. A method of detecting tissue damage before it is visible on a patient's skin, comprising: measuring a plurality of sub-epidermal moisture (SEM) values at a single location at each of a plurality of incremental times, calculating a SEM delta value for each incremental time, fitting a curve to a predetermined number of the most-recent SEM delta values, calculating a curvature of the fitted curve, comparing this curvature to a threshold value, and determining that there is tissue damage if the curvature exceeds the threshold value.

Having now generally described the invention, the same will be more readily understood through reference to the following examples that are provided by way of illustration, and are not intended to be limiting of the present disclosure, unless specified.

### EXAMPLES

### Example 1: SEM Delta Trends in Heels of Patients is Indicative of Pressure Ulcer Onset

SEM measurements are taken over time at the heels of patients using an apparatus according to the present disclosure, prior to any visual diagnosis of pressure ulcers at the heel. At each time point, each of the patients is directed to have toes pointed away from the body and rotated outwards toward the lateral side of the body. A sensor of the apparatus is placed on the medial side of the heel. The sensor is adjusted for full contact with the heel, and multiple measurements are taken around the back of the heel in a curve. Each of the SEM measurements is converted to a SEM delta value by subtracting from the measurement a reference SEM value obtained from another body part of the same patient that is not experiencing external pressure or mechanical forces. The resulting SEM delta values in a single day are averaged and plotted for each patient.

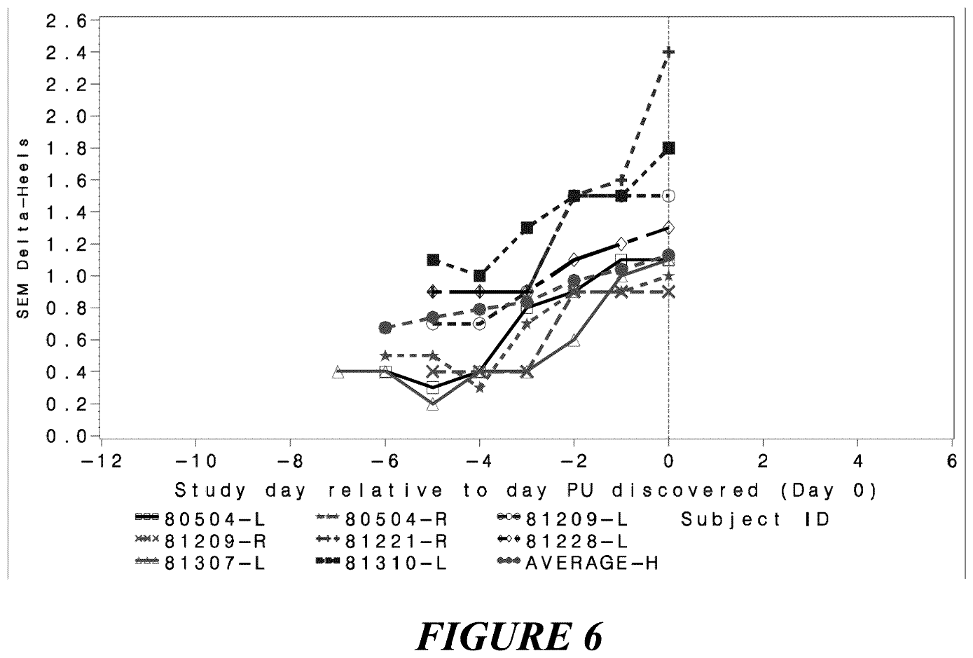

FIG. 6 illustrates trends of SEM delta values for seven (7) patients prior to a pressure ulcer diagnosis at one or both of their heels. Trends across different patients are time-shifted to align to Day 0 as the pressure ulcer diagnosis event. A reference SEM delta curve (“AVERAGE-H”) is generated by averaging the SEM delta values trends of all patients (n=20) that are eventually visually diagnosed with a heel pressure ulcer. As shown in FIG. 6, the SEM delta values of these seven patients exhibit a spike in magnitude two (−2) to four (−4) days prior to the visual diagnosis compared to the reference curve. For these patients, a steeper slope of the SEM delta value trend compared to the reference curve is indicative of early onset of a pressure ulcer before any visual detection.

We claim:

1. A method of detecting tissue damage before it is visible on a patient's skin, comprising:
measuring a plurality of sub-epidermal moisture (SEM) values at a single location at incremental times,
calculating a slope between the latest SEM value and the immediately prior SEM value,
comparing this slope to a threshold value, and
determining that there is tissue damage if the slope exceeds the threshold value.

2. A method of detecting tissue damage before it is visible on a patient's skin, comprising:
measuring a plurality sub-epidermal moisture (SEM) values at a plurality of locations at incremental times,
calculating a delta value for the plurality of SEM values for each time,
calculating a slope between the latest delta value and the immediately prior delta value,
comparing this slope to a threshold value, and
determining that there is tissue damage if the slope exceeds the threshold value.

3. A method of detecting tissue damage before it is visible on a patient's skin, comprising:
measuring a plurality of sub-epidermal moisture (SEM) values at a single location at each of a plurality of incremental times,
calculating a SEM delta value for each incremental time,
fitting a curve to a predetermined number of the most-recent SEM delta values,
calculating a curvature of the fitted curve,
comparing this curvature to a threshold value, and
determining that there is tissue damage if the curvature exceeds the threshold value.
